(19) United States
(12) Patent Application Publication
(10) Pub. No.: US 2011/0288176 A1
(43) Pub. Date: Nov. 24, 2011

(54) ETHACRYNIC ACID-CONTAINING COMPOSITION FOR PREVENTION OR TREATMENT OF TRANSGLUTAMINASE-RELATED DISEASES AND METHOD FOR PREVENTION OR TREATMENT OF TRANSGLUTAMINASE-RELATED DISEASES USING THE SAME

(76) Inventors: Soo Youl Kim, Seoul (KR); Chang Hoon Lee, Gyeonggi-do (KR); Byung Il Lee, Gyeonggi-do (KR); Kyung Chae Jeong, Gyeonggi-do (KR)

(21) Appl. No.: 12/920,406

(22) PCT Filed: Nov. 27, 2009
(86) PCT No.: PCT/KR2009/007057

§ 371 Date: Oct. 20, 2010

(30) Foreign Application Priority Data
Nov. 27, 2008 (KR) 10-2008-0118688

(51) Int. Cl. A61K 31/192; A61P 35/00; A61P 25/00; C12N 9/99; A61P 19/02; A61P 37/06; A61P 25/28; A61P 25/16; A61P 29/00; A61P 35/04

(52) U.S. Cl. 514/571; 435/184

(57) ABSTRACT

The present invention relates to ethacrynic acid and a transglutaminase inhibitor comprising a pharmaceutically acceptable salt of ethacrynic acid; more particularly, to ethacrynic acid for inhibiting the activity of transglutaminase involved in the pathogenesis of diseases in cases of altered expression, a transglutaminase inhibitor comprising a pharmaceutically acceptable salt of ethacrynic acid, and to a novel use of thereof. According to the present invention, there are provided ethacrynic acid, a transglutaminase inhibitor comprising active components of a pharmaceutically acceptable salt of ethacrynic acid and a transglutaminase inhibition method. The present ethacrynic acid, transglutaminase inhibitor comprising a pharmaceutically acceptable salt of ethacrynic acid or ethacrynic acid itself, and transglutaminase inhibition method provide a safer application without side effects to a patient having a disease where altered expression of transglutaminase is observed, thereby providing the inhibitory effect on transglutaminase.

### TECHNICAL FIELD

The present invention relates to a transglutaminase inhibitor comprising ethacrynic acid or a pharmaceutically acceptable salt thereof; more particularly, to a transglutaminase inhibitor comprising ethacrynic acid or a pharmaceutically acceptable salt thereof for inhibiting the activity of transglutaminase which is involved in the pathogenesis of diseases in cases of altered expression, and a novel use thereof.

### BACKGROUND ART

Transglutaminase is an enzyme that cross-links protein molecules, and is widely distributed in nature, having been found in various animals, plants, and microorganisms. When transglutaminase acts on protein molecules, it catalyzes protein cross-linking via acyl-transfer reaction, cross-linking reaction, and deamination, and this cross-linkage has unique effects on gelation capability, thermal stability, water-holding capacity, etc. Transglutaminase is a protective enzyme which is responsible for blood clotting in response to tissue injury under normal conditions. However, this enzyme is also reported to play an important role in the pathological mechanism of various diseases in the absence of regulatory-control in the level of expression thereof (review article. Soo-Youl Kim: New Target Against Inflammatory Diseases Transglutaminase 2. Archivum Immunologiae & Therapiae Experimentalis 52, 332-337, 2004).

The expression of transglutaminase increases particularly upon the occurrence of various inflammatory diseases, including chronic inflammatory diseases such as degenerative arthritis, diabetes, inflammatory myositis, atherosclerosis, stroke, liver cirrhosis, malignant breast cancer, Alzheimer's disease, Parkinson's disease, Huntington's disease, meningitis, inflammatory gastric ulcer, and septicemia. Also, transglutaminase is observed to increase in expression level, along with NF-κB, when cancer enters metastasis or changes into chemo-resistance or radio-resistance (Soo-Youl Kim. Transglutaminase 2 in inflammation. Front Biosci. 11, 3026-3035, 2006).

The relationship between transglutaminase and chemo-resistance in cancer has remained unclear so far. However, when the expression of transglutaminase was suppressed in chemoresistant breast cancer cells, the cancer cells highly susceptible to chemicals, and finally died (Antonyak et al., Augmentation of tissue tansglutaminase expression and activation by epidermal growth factor inhibit doxorubicin-induced apoptsis in human breast cancer cells. J Biol. Chem. 2004 Oct. 1; 279(40):41461-7; Dae-Seok Kim et al. Reversal of Drug Resistance in Breast Cancer Cells by Transglutaminase 2 Inhibition and Nuclear Factor-KB Inactivation. Cancer Res. 2006. in press).

Additionally, there is an additional strong reason for suppressing the activity of transglutaminases as the etiological mechanism for which the activation of transglutaminases is responsible is elucidated at the molecular level (Key Chung Park, Kyung Cheon Chung, Yoon-Seong Kim, Jongmin Lee, Tong H. Joh, and Soo-Youl Kim. Transglutaminase 2 induces nitric oxide synthesis in BV-2 microglia. Biochem. Biophys. Res. Commun. 323, 1055-1062, 2004; Jongmin Lee, Yoon-Seong Kim, Dong-Hee Choi, Moon S. Bang, Tay R. Han, Tong H. Joh, and Soo-Youl Kim. Transglutaminase 2 induces NF-κB activation via a novel pathway in BV-2 microglia. J. Biol. Chem. 279, 53725-53735, 2004).

The mechanism of inflammation has not yet been fully clarified, but inflammation is largely attributable to NF-κB activation. NF-κB is known to be activated by kinases in signal transduction pathways. However, NF-κB was also found to be activated independently of kinases, thereby negating the function of kinase inhibitors (Tergaonkar et al., IkappaB kinase-independent IkappaBalpha degradation pathway: functional NF-kappaB activity and implications for cancer therap. Mol Cell Biol. 2003 November; 23(22):8070-83).

The present inventors found that NF-κB and transglutaminase are highly expressed in inflammation, and elucidated its mechanism in the inflammation model. In the study, it was reported that transglutaminase activates NF-κB independently of the activation of linases (IKK, NAK), by inducing cross-linking I-κBα (Jongmin Lee, et al. Transglutaminase 2 induces NF-κB activation via a novel pathway in BV-2 microglia. J. Biol. Chem. 279, 53725-53735, 2004). Transglutaminase is a calcium-dependent enzyme, which can activate NF-κB only at an elevated intracellular level of calcium.

Upon inflammation, the activation of the transcriptional factor NF-κB leads to an increase in the expression not only of inflammatory factors including transglutaminases, but also of its inhibitor I-κBα. Continuous NF-κB activation is inhibited by I-κBα under normal conditions, but continues in chronic inflammatory diseases. Interestingly, TNF-α or LPS (lipopolysaccharide)-induced NF-κB activation gives rise to transglutaminase expression. Thus, aberrantly activated transglutaminases in inflammatory cells are expected to activate NF-κB directly or to further maintain activated NF-κB, thereby playing a key role in inflammation maintenance. On the basis of this theory, a feedback loop was suggested. In general, it is supposed that NF-κB activation produces I-κBα to suppress NF-κB and induces the transglutaminase inhibitor, thereby preventing the feedback loop of NF-κB activation by transglutaminase. However, if homeostasis is disrupted, continuous NF-κB activation by transglutaminase may cause inflammation by inflammatory cells. In addition, this vicious cycle may be a main cause of cancer metastasis and chemoresistance (Jongmin Lee, et al. Transglutaminase 2 induces NF-κB activation via a novel pathway in BV-2 microglia. J. Biol. Chem. 279, 53725-53735, 2004).

Therefore, a transglutaminase inhibitor may play a crucial role in breaking the continuous cycle of NF-κB so as to improve inflammatory diseases, on which the steroid-substituting effect from recombinant peptides as transglutaminase inhibitor proposed by the present inventors is based (Sohn, J., Kim, T.-I., Yoon, Y.-H., and kim, S.-Y.: Transglutaminase Inhibitor: A New Anti-Inflammatory Approach in Allergic Conjunctivitis. J. Clin. Invest. 111, 121-8, 2003).

Amine compounds are known to inhibit transglutaminase activity. Representative of the transglutaminase inhibitors are cystamine (nature Genetics 18, 111-117, 1998; Nature Medicine 8, 143-149, 2002) and putrescine. In addition to the amine compounds, other chemical inhibitors, such as monodansylcadaverine (J. Med. Chem. 15, 674-675, 1972), w-dibenzylaminoalkylamine (J. Med. Chem. 18, 278-284, 1975), and 3-halo-4,5-dihydroisoxazole (Mol. Pharmacol. 35, 701-706, 1989), were developed, but are reported to be so toxic as to non-specifically inhibit other enzymes in vivo.

Therefore, there is a need for safe and effective transglutaminase-specific inhibitors. Recently, Sohn et al. have succeeded in obtaining the same effect from recombinant peptides as steroidal drugs for the inflammation of allergic conjunctivitis to ragweed in a guinea pig model (Sohn, J., Kim, T.-I., Yoon, Y.-H., and Kim, S.-Y.: Transglutaminase Inhibitor: A New Anti-Inflammatory Approach in Allergic Conjunctivitis. J. Clin. Invest. 111, 121-8, 2003).

In this regard, anti-flammin protein (PLA2 inhibitor) or elafin protein (very strong transglutaminase substrate, Nara, K., et al. 1994. Elastase inhibitor elafin is a new type of proteinase inhibitor which has a transglutaminase-mediated anchoring sequence termed “cementoin”. J Biochem (Tokyo). 115:441-448)-derived synthetic peptides which mimic the catalytic site of transglutaminase were used. The expression of transglutaminases increases particularly upon the occurrence of various inflammatory diseases, including chronic inflammatory diseases such as degenerative arthritis, diabetes, autoimmune myositis, arteriosclerosis, stroke, liver cirrhosis, malignant breast cancer, meningitis, inflammatory gastric ulcer, and septicemia.

In addition to the above-mentioned compounds, other chemical inhibitors were developed, but are reported to be so toxic as to non-specifically inhibit other enzymes in vivo. Effective as they are in inhibiting transglutaminase, peptide inhibitors developed prior to the present invention (Korean Patent Application No. 10-2006-98921) still have a lot of problems awaiting solutions in terms of production cost and safe practice.

Ethacrynic acid (2-[2,3-dichloro-4-(2-methylidenebuanoyl)phenoxy]acetic acid) is a loop diuretic used to treat high blood pressure and the swelling caused by diseases like congestive heart failure, liver failure, and kidney failure. Unlike the other loop diuretics, ethacrynic acid is not a sulfonamide and thus, its use is not contraindicated in those with sulfa allergies. Ethacrynic acid is a phenoxyacetic acid derivative containing a ketone and a methylene group.

### DISCLOSURE

### Technical Problem

Accordingly, the present inventors selected drugs which have been recognized as being safe for commercialization and of which the mechanisms are not well known, and then investigated their mechanisms and tried to find their novel uses. Among them, the present inventors have screened materials useful as a transglutaminase inhibitor. As a result, they found that ethacrynic acid has a potent inhibitory activity against transglutaminase, thereby completing the present invention.

### Technical Solution

It is an object of the present invention to provide a transglutaminase inhibitor comprising ethacrynic acid or a pharmaceutically acceptable salt thereof.

Further, it is another object of the present invention to provide a method for inhibiting transglutaminase using ethacrynic acid or a pharmaceutically acceptable salt thereof as an active ingredient.

It is still another object of the present invention to provide a pharmaceutical composition using ethacrynic acid, or a pharmaceutically acceptable salt thereof for the treatment of diseases caused by the increased activation of transglutaminase.

It is still another object of the present invention to provide a method for treating diseases caused by the increased activation of transglutaminase using ethacrynic acid or a pharmaceutically acceptable salt thereof.

### Advantageous Effects

According to the present invention, provided are a transglutaminase inhibitor comprising ethacrynic acid or a pharmaceutically acceptable salt thereof as an active ingredient, and a method for inhibiting transglutaminase.

The novel method for inhibiting transglutaminase using ethacrynic acid or a pharmaceutically acceptable salt thereof according to the present invention is safely applied to patients who suffer from the diseases caused by the altered expression of transglutaminase, thereby obtaining an inhibitory effect against transglutaminase without causing side-effects.

### DESCRIPTION OF DRAWINGS

FIG. 1 is a graph showing the result of in vitro assay for inhibitory effect of ethacrynic acid on transglutaminase, in which the transglutaminase-catalyzed reaction between [1,4,-¹⁴C]putrescine and succinylated casein is measured, resulting in that ethacrynic acid acts to inhibit the activity of transglutaminase.

### BEST MODE

In accordance with an aspect, the present invention relates to ethacrynic acid, a pharmaceutically acceptable salt thereof, or a transglutaminase inhibitor comprising the same, and a method for inhibiting the activity of transglutaminase using ethacrynic acid as an active ingredient.

Ethacrynic acid (2-[2,3-dichloro-4-(2-methylidenebuanoyl)phenoxy]acetic acid) is a loop diuretic used to treat high blood pressure and the swelling caused by diseases like congestive heart failure, liver failure, and kidney failure. Unlike the other loop diuretics, ethacrynic acid is not a sulfonamide, and thus its use is not contraindicated in those with sulfa allergies. Ethacrynic acid is a phenoxyacetic acid derivative containing a ketone and a methylene group.

Ethacrynic acid has a molecular formula of C₁₃H₁₂Cl₂O₄, a molecular weight of 303.14, and the following structural formula.

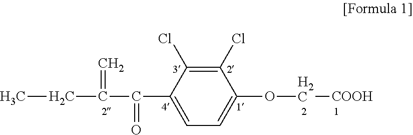

In the present invention, synthesized ethacrynic acid may be used, and it may be obtained by synthesis, isolation and purification according to the known method. In addition, the ethacrynic acid may be directly prepared or obtained from commercial sources.

Further, the transglutaminase inhibitor according to the present invention may include a pharmaceutically acceptable salt of ethacrynic acid.

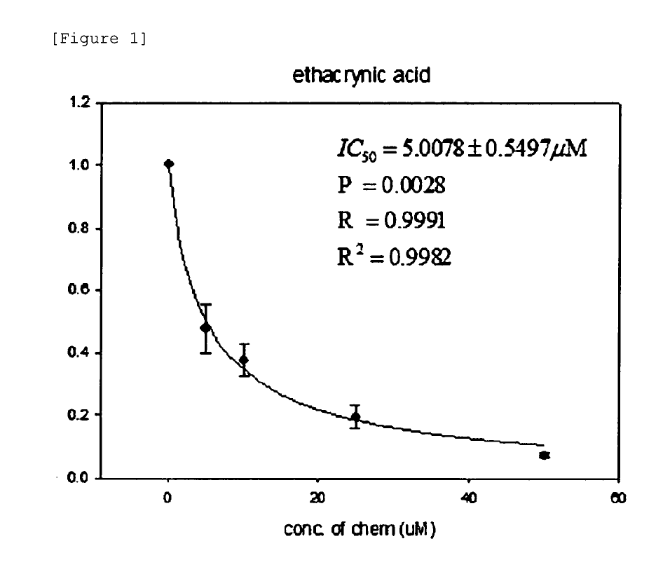

In a specific embodiment of the present invention, the present inventors measured transglutaminase-catalyzed reaction between [1,4, —C] putrescine and succinylated casein. It was found that ethacrynic acid acts to inhibit the activity of transglutaminase. In in vitro experiments for transglutaminase-catalyzed reaction between ¹⁴C-labelled putrescine and succinylated casein, it was found that a higher concentration of ethacrynic acid leads to a poorer activity of transglutaminase (FIG. 1). Consequently, it can be seen that ethacrynic acid is a transglutaminase inhibitor, and ethacrynic acid can reduce the increased activity of transglutaminase, even upon the overexpression of transglutaminase.

In accordance with another aspect, the present invention relates to a pharmaceutical composition for the prevention and treatment of diseases caused by the increased activation of transglutaminase, comprising ethacrynic acid or a pharmaceutically acceptable salt thereof, and to a method for treating the diseases using ethacrynic acid or a pharmaceutically acceptable salt thereof.

The term “prevention”, as used herein, means all of the actions in which the occurrence of any disease caused by the increased activation of transglutaminase is restrained or retarded by the administration of the pharmaceutical composition containing ethacrynic acid or a pharmaceutically acceptable salt thereof. The term “treatment”, as used herein, means all of the actions in which any disease caused by the increased activation of transglutaminase has taken a turn for the better or been modified favorably by the administration of the pharmaceutical composition.

In the present invention, the diseases caused by increased activation of transglutaminase include all diseases that are incurred as transglutaminase activity increases, for example, upon the overexpression of transglutaminase, and are particularly exemplified by neurological diseases, inflammatory diseases, and cancers.

The expression of transglutaminases increases particularly upon the occurrence of various inflammatory diseases, including chronic inflammatory diseases such as degenerative arthritis, autoimmune myositis, atherosclerosis, stroke, liver cirrhosis, malignant breast cancer, meningitis, inflammatory gastric ulcer, and septicemia.

Typical of neurological diseases are central nervous system diseases, which are associated with the death or injury of the central nervous system, such as Alzheimer's disease, multi-infarct dementia, a mixed Alzheimer/multi-infarct dementia, Parkinson's disease, hypothyroidism, alcohol-related dementia, and Huntington's diseases. These diseases are characterized by confusion, disorientation and personality disintegration with main syndromes of cognitive dysfunction, language impairment, dysfunctions in judgment, inference, temporal and spatial adaptation and learning, finally leading to the death of afflicted patients. Of them, the diseases caused by increased activation of transglutaminase, e.g., the overexpression of transglutaminase in nerve tissues, are targets of the pharmaceutical composition according to the present invention. Particularly, the pharmaceutical composition of the present invention is useful in the treatment of Huntington's disease, which is associated with the over-expression of transglutaminase in the brain (Nature Medicine, Vol 8. Number 2, February 2002 pp 143-149), Alzheimer's disease, which is associated with the over-expression of transglutaminase in the cerebellum and cerebral cortex (The Journal of Biological Chemistry, Vol. 274. No. 43. Issue Of October 22, pp 30715-30721), and Parkinson's disease, which is associated with transglutaminase-induced α synuclein aggregation (PNAS, Feb. 18, 2003, Vol. 100, no. 4, pp 2047-2052), but are not limited thereto. The present invention is applicable to the treatment of all diseases caused by the overexpression of transglutaminase in nerve tissues.

As for cancers, these are found to significantly increase in the level of expression of transglutaminase upon metastasis or entry into chemo- or radio-resistance. Thus, the suppression of transglutaminase arises as a key in the prevention and treatment of cancers. Concrete examples of the cancers, which can be prevented or treated using the pharmaceutical composition containing ethacrynic acid of the present invention, include cancers with increased transglutaminase, specifically, large intestine cancer, small intestine cancer, rectal cancer, anal cancer, esophageal cancer, pancreatic cancer, stomach cancer, kidney cancer, uterine cancer, breast cancer, lung cancer, lymphoma, thyroid cancer, prostatic carcinoma, leukemia, skin cancer, colon cancer, encephaloma, bladder cancer, ovarian cancer, and gallbladder carcinoma, but are not limited thereto.

The composition comprising ethacrynic acid or a pharmaceutically acceptable salt thereof and the treatment method of the present invention can be applied to mammals that may suffer from diseases due to increased activation of transglutaminase, including cattle, horses, sheep, pigs, goats, camels, antelopes, dogs, and cats, as well as humans.

The ethacrynic acid of the present invention or pharmaceutically acceptable salt thereof or the pharmaceutical composition using the same may be used alone or in combination with other pharmaceutical compositions.

The pharmaceutical composition comprising ethacrynic acid or a pharmaceutically acceptable salt thereof may be loaded into a capsule containing ethacrynic acid without an excipient or together with a fine solid carrier and/or a liquid carrier. If necessary, the resultant may be molded into preferred formulations. Examples of the suitable carriers include starch, water, brine, ethanol, glycerol, Ringer's solution, and dextrose solutions. Reference may be made to the literature (Remington's Pharmaceutical Science, 19th Ed., 1995, Mack Publishing Company, Easton Pa.) upon formulation of the pharmaceutical composition.

The pharmaceutical composition comprising ethacrynic acid or a pharmaceutically acceptable salt thereof of the present invention may be prepared into any dosage form, either oral or non-oral formulation, containing ethacrynic acid or a pharmaceutically acceptable salt thereof as an active ingredient. Non-oral dosage forms may be injections, coatings, and sprays such as aerosols, with preference for injections or sprays such as aerosols. Also preferable are oral dosage forms.

Examples of the oral dosage forms suitable for the pharmaceutical composition of the present invention include tablets, troches, lozenges, aqueous or emulsive suspensions, powder, granules, emulsions, hard or soft capsules, syrups, and elixirs. For formulation such as tablets and capsules, useful are a binder such as lactose, saccharose, sorbitol, mannitol, starch, amylopectin, cellulose and gelatin, an excipient such as dicalcium phosphate, a disintegrant such as corn starch and sweet potato starch, a lubricant such as magnesium stearate, calcium stearate, sodium stearylfumarate, and polyethylene glycol wax. For capsules, a liquid carrier such as oil may be further used in addition to the above-mentioned compounds.

For non-oral administration, the pharmaceutical composition of the present invention may be formulated into injections for subcutaneous, intravenous, or intramuscular routes, suppositories, or sprays inhalable via the respiratory tract, such as aerosols. Injectable preparations may be obtained by dissolving or suspending ethacrynic acid, a pharmaceutically acceptable salt thereof, or a derivative thereof, together with a stabilizer or a buffer, in water and packaging the solution or suspension in ampules or vial units. Suppositories are typically made of a suppository base, such as cocoa butter and another glyceride, or a therapeutic laxative. For sprays, such as aerosol, a propellant for spraying a water-dispersed concentrate or wetting powder may be used in combination with an additive.

The pharmaceutical composition of the present invention may be administered via typical routes, such as rectal, local, intravenous, intraperitoneal, intramuscular, intraarterial, transdermal, intranasal, inhalational, intraocular, and subcutaneous routes. Non-oral administration means administration modes including intravenous, intramuscular, intraperitoneal, intrasternal, transdermal and intraarterial routes. For administration via non-oral routes, the pharmaceutical composition comprising ethacrynic acid in a desired purity is preferably mixed with a pharmaceutically acceptable carrier, that is, a carrier being non-toxic at dosage concentrations and amounts, and compatible with other ingredients, and then formulated into a unit dosage form. In particular, it is required to exclude oxidants and other compounds known to be hazardous to the human body.

The ethacrynic acid or pharmaceutically acceptable salt thereof of the present invention may be administered along with at least one pharmaceutically acceptable excipient as a pharmaceutical composition. It will be obvious to those skilled in the art that when the pharmaceutical composition of the present invention is administered to human patients, the total daily dose should be determined through appropriate medical judgment by a physician. The therapeutically effective amount for patients may vary depending on various factors well known in the medical art, including the kind and degree of the response to be achieved, concrete compositions according to whether other agents are used therewith or not, the patient's condition such as age, body weight, state of health, sex, and diet, the frequency, time and route of administration, the secretion rate of the composition, the time period of therapy, etc. For agents suitable for use in the art, reference may be made to the literature (Remington's Pharmaceutical Science, 19^{th} Ed., 1995, Mack Publishing Company, Easton Pa.). Accordingly, the effective dosage of ethacrynic acid or a pharmaceutically acceptable salt thereof, suitable for the purpose of the present invention, is preferably determined with reference to the above-mentioned considerations.

### MODE FOR INVENTION

Hereinafter, a better understanding of the present invention may be obtained through the following examples which are set forth to illustrate, but are not to be construed as the limit, of the present invention.

### Example 1

### In Vitro Assay for Inhibition of Transglutaminase Activity

To measure the inhibitory activity of ethacrynic acid, the transglutaminase-catalyzed reaction between [1,4,-¹⁴C] putrescine and succinylated casein were observed.

Succinylated casein was purchased from Calbiochem (Cat. No. 573464), and 1 g of the powder was dissolved in 50 ml of a reaction buffer solution containing 5 mM DTT (0.1 M Tris-acetate (pH 8.0), 10 mM CaCl₂, 0.15 M NaCl, 1.0 mM EDTA). This solution was stored in a deep freezer for further use. [1,4-¹⁴C] Putrescine dihydrochloride was purchased from GE Healthcare (Cat. No. CFA301), and the stock solution was diluted with distilled water to yield a radiological dose of 5 μCi/ml. Transglutaminase 2 was purchased from Sigma-Aldrich (Cat. No. T5398), and diluted with distilled water to yield a final concentration of 1 unit/ml. Ethacrynic acid was dissolved in DMSO at a concentration of 10 mM to prepare its stock solution, and the stock solution was diluted with DMSO to prepare solutions having various concentrations.

450 μl of succinylated casein solution and 50 μl of [1,4-¹⁴C] putrescine dihydrochloride solution were mixed together to prepare a substrate solution. 96 μl of the reaction buffer, 3 μl of the stock solution of ethacrynic acid, and 1 μl of the stock solution of transglutaminase were mixed together to prepare each sample, followed by incubation at 37° C. for 10 min. 500 μl of the substrate solution β and 100 μl of sample solution were mixed well, and the mixture was incubated at 37° C. for 2 hrs, before termination with 4.5 ml of cold (4° C.) 7.5% TCA. The final solution was stored at 4° C. for 1 hr. The TCA-protein precipitates were filtered through a GF/glass fiber filter, washed with 25 ml of cold 5% TCA, and dried. Radioactivity of cross-linked protein was measured using a (β-counter (Beckman Coulter), and compensated by the activity of DMSO-control group as a standard. The activity of transglutaminase was represented by the measured values. The assay was repeated three times under the same conditions, and the results are shown in the following Table 1.

TABLE 1

| | | | | | SD |
|---|---|---|---|---|---|
| | | | | | (standard |
| Concentration | Assay 1 | Assay 2 | Assay 3 | Mean | deviation) |
| 0.0 μM | 1.0000 | 1.0000 | 1.0000 | 1.0000 | 0.0000 |
| 5 μM | 0.5667 | 0.4572 | 0.4148 | 0.4796 | 0.0783 |
| 10 μM | 0.4319 | 0.3654 | 0.3281 | 0.3752 | 0.0526 |
| 25 μM | 0.2337 | 0.1812 | 0.1656 | 0.1935 | 0.0357 |
| 50 μM | 0.0763 | 0.0799 | 0.0645 | 0.0736 | 0.0080 |

In addition, the mean value was depicted in terms of concentration of ethacrynic acid. IC₅₀ values were calculated by a general nonlinear regression method, determined as 4.0078 (±0.5479) μM.

The relative inhibition activities of ethacrynic acid against transglutaminase are depicted in FIG. 1. As shown in FIG. 1, it can be seen that the activity of transglutaminase was inhibited in an ethacrynic acid concentration-dependent manner.

### INDUSTRIAL APPLICABILITY

The present invention relates to a transglutaminase inhibitor comprising ethacrynic acid or a pharmaceutically acceptable salt thereof. The present invention provides a pharmaceutical composition and a treatment method for inhibiting the activity of transglutaminase, which are useful for the treatment of diseases caused by the altered expression of transglutaminase, such as neurological diseases and cancers. Therefore, the transglutaminase inhibitor comprising ethacrynic acid or a pharmaceutically acceptable salt thereof, the method for inhibiting transglutaminase, and the treatment method according to the present invention is safely applied to patients who suffer from the diseases caused by the altered expression of transglutaminase, thereby obtaining an inhibitory effect against transglutaminase without causing side-effects.

1. A pharmaceutical composition for the treatment or prevention of diseases caused by the increased activation of transglutaminase, comprising ethacrynic acid or a pharmaceutically acceptable salt thereof.

2. The pharmaceutical composition according to claim 1, wherein the diseases are inflammatory diseases, cancers, or neurological diseases.

3. The pharmaceutical composition according to claim 2, wherein the composition has an inhibitory effect on cancer metastasis.

4. The pharmaceutical composition according to claim 2, wherein the inflammatory disease is selected from the group consisting of chronic inflammatory diseases including degenerative arthritis, septicemia, and autoimmune myositis.

5. The pharmaceutical composition according to claim 2, wherein the neurological disease is selected from the group consisting of Alzheimer's disease, Huntington's disease, and Parkinson's disease.

6. A method for inhibiting a transglutaminase activity using ethacrynic acid or a pharmaceutically acceptable salt thereof.

7. A method for the treatment or prevention of a disease caused by the increased activation of transglutaminase, comprising the step of administering the pharmaceutical composition of claim 1.

8. The method according to claim 7, wherein the diseases are inflammatory diseases, cancers, or neurological diseases.

9. The method according to claim 8, wherein the method has an inhibitory effect on cancer metastasis.

10. The method according to claim 8, wherein the inflammatory disease is selected from the group consisting of chronic inflammatory diseases including degenerative arthritis, septicemia, and autoimmune myositis.

11. The method according to claim 8, wherein the neurological disease is selected from the group consisting of Alzheimer's disease, Huntington's disease, and Parkinson's disease.
